(19) United States
(12) Patent Application Publication
(10) Pub. No.: US 2004/0248772 A1
(43) Pub. Date: Dec. 9, 2004

(54) Anticancer compositions

(76) Inventor: Akikuni Yagita, Tokyo (JP)

Correspondence Address: KILYK & BOWERSOX, P.L.L.C., 53 A EAST LEE STREET, WARRENTON, VA, 20186, US

(21) Appl. No.: 10/494,837

(22) PCT Filed: Nov. 5, 2002
(86) PCT No.: PCT/JP02/11512

(30) Foreign Application Priority Data
Nov. 6, 2001 (JP) 2001341115
Feb. 22, 2002 (JP) 200246694
Jun. 28, 2002 (JP) 2002191474

(51) Int. Cl. A61K 38/17

(52) U.S. Cl. 514/2

(57) ABSTRACT

An object of the present invention is to provide an anticancer composition. More specifically, the anticancer composition according to the present invention was successfully provided by newly finding the fact that a composition comprising NGB or IMMUTOL, a yeast-derived ingredient having β1,3/1,6 glucan structure each, is an unprecedentedly effective IL-12 inducer and further newly discovering that the composition can hold promise of NK and NKT cell activating capabilities and tumor angiogenesis inhibiting capability as a result of oral administration of IMMUTOL (trade name), a yeast-derived ingredient having β1,3/1,6 glucan structure.

This application claims the benefit of priority from Japanese Patent Application Nos. 2001-341115, 2002-046694 and 2002-191474, of which full contents are incorporated herein by reference.

### TECHNICAL FIELD

The present invention relates to provision of a new anticancer composition. More specifically, the present invention relates to IL-12 inducer comprising NBG or IMMUTOL (trade name), a yeast-derived ingredient having β1,3/1,6 glucan structure each. Further, the present invention relates to an anticancer composition that can hold promise of NK and NKT cell activating capabilities and tumor angiogenesis inhibiting capability by oral administration of IMMUTOL (trade name), a yeast-derived ingredient having β1,3/1.6 glucan structure.

### BACKGROUND OF THE INVENTION

In selecting substances useful for prevention or treatment of cancer (malignant neoplasms), emphasis has hitherto been placed on their direct effect on cancer cells. While immunostimulators have been recognized as useful for cancer treatment, all compounds obtained as immunostimulators are feeble in their anticancer effect, leaving sufficient cancer treatment effect unattained both by immunotherapy alone and combination of immunotherapy and chemical therapy.

The present inventor, MD. Yagita, noting previously the usefulness of substances inducing interleukin 12 (IL-12) in vivo as an epoch-making method in cancer treatment, discovered that processed shiitake mycelium has that function, thus established a cancer treatment method that might be described as “novel immunotherapy for cancer”(NITC). IL-12 has been unusable as an anticancer drug despite its anticancer effect, because of the fact that patients are unable to endure treatment due to its side effects when IL-12 itself is directly administered in vivo. However, the preparation containing the processed shiitake mycelium reported by Yagita achieved outstanding curing- and life-prolonging effects in cancer treatment. That is, Yagita achieved the purpose of cancer treatment by administering the processed shiitake mycelium in effective amounts sufficient to induce IL-12 in vivo (Japanese Patent Application Laid-Open Publication No. 1998-139670).

The IL-12 has activating and augmenting effects on killer T cell through the route of TNFα→IFNγ→IL-12→CTL activation. That is, augmentation of IL-12 production holds promise of anticancer effect by activating and augmenting killer T cell.

Yagita also reported, aside from the system of IL-12 production augmentation, that NKT cell activation is useful for anticancer effect. Taniguchi et al discovered a specific glicolipid antigen recognized by a specific T cell antigen receptor (TCR), Vα24Vβ11, carried in NKT cell, and reported that this antigen is a galactosylceramide. Further, they proved that, in a cancer-bearing mouse administered with α galactosylceramide, NKT cell is activated and metastasis suppressed, although the cancer disappearance is not observed.

It is reported that there is NK cell antigen receptor (NKR-P1; natural killer receptor P1) as another receptor in NKT cell (Feature Article—Basics and Clinicals of NKT Cell: Saishin Igaku Vol. 55, No. 4, 2000, P.818-823). Yagita found that NKR-P1 also participates in NKT cell activation and that this activation enhances anticancer effect.

It is reported that NK cell also plays a part in anticancer effect in vivo. However, the facts were proved by Yagita that NK cell activation and clinical anticancer effect are not correlated and that the amount of IL-12 production induced and NK cell activation exhibit a completely inverse correlation. Therefore, the credibility of NK cell playing a part in anticancer effect in human has been questioned.

MD. Yagita has studied hitherto various in vivo IL-12 inducers, and found a new IL-12 inducer (ILY registered trademark: Orient Cancer Therapy Co., Ltd., trade name) derived from bracket fungus mycelium ,taking cancer cycle into consideration. Discovering that mushroom mycelium containing β1,3/1,6 glucan has antitumor effect and that its antitumor property results from cytokine (IL-12) activating totally Thl immunity, MD. Yagita has applied for patents for new use of products such as trade names AHCC, ILX, ILY, Krestin and SPG.

### SUMMARY OF THE INVENTION

It is one object of the present invention to provide a further useful IL-12 inducer, and more particularly to provide a more effective IL-12 inducer even in the case of the cancer that has progressed to a serious stage (progressive cancer, terminal cancer). It is another object of the present invention to provide an anticancer composition that can hold promise of NK and NKT cell activating capabilities and tumor angiogenesis inhibiting capability by oral administration of IMMUTOL (trade name), a yeast-derived ingredient having β1,3/1.6 glucan structure.

The present invention found as a result of study on yeasts as new substances, that a composition comprising NBG or IMMUTOL (trade name), a yeast-derived ingredient having β1,3/1,6 glucan structure each, is an unprecedentedly effective IL-12 inducer and that such a composition is an anticancer composition that can hold promise of NK and NKT cell activating capabilities and tumor angiogenesis inhibiting capability by oral administration of IMMUTOL (trade name), a yeast-derived ingredient having β1,3/1.6 glucan structure, thus successfully provided an anticancer composition according to the present invention.

That is, the present invention consists of:

1. An IL-12 inducer comprising NBG or IMMUTOL (trade name), each of which is a yeast-derived ingredient having β1,3/1,6 glucan structure.

2. An NK cell and/or NKT cell activator comprising IMMUTOL (trade name), which is a yeast-derived ingredient having β1,3/1,6 glucan structure.

3. The inducer according to 1 or the activator according to 2, wherein NBG or IMMUTOL, each of which is a yeast-derived ingredient having β1,3/1,6 glucan structure, is ingested orally in vivo from 10 to 2000 mg/kg of body weight/day in amounts.

4. The health aid food preparations intended for oral ingestion according to 1 to 3.

5. An anticancer composition, comprising IMMUTOL (trade name), which is a yeast-derived ingredient having β1,3/1,6 glucan, being able to hold promise of IL-12 inducing capability, NK cell activating capability, NKT cell activating capability, and tumor angiogenesis inhibiting capability.

6. A cancer treatment drug comprising the composition according to 5, wherein the form of administration is oral.

7. A cancer treatment method, wherein IMMUTOL (trade name), which is a yeast-derived ingredient having β1,3/1,6 glucan structure, is ingested, with IL-12 inducing capability as treatment marker.

8. A cancer treatment method, wherein IMMUTOL (trade name), which is a yeast-derived ingredient having β1,3/1,6 glucan structure, is ingested, with NK cell activating capability and/or NKT cell activating capability as treatment marker.

9. A cancer treatment method, wherein IMMUTOL (trade name), which is a yeast-derived ingredient having β1,3/1,6 glucan structure, is ingested, with tumor angiogenesis inhibiting capability as treatment marker.

10. A cancer treatment method, wherein IMMUTOL (trade name), which is a yeast-derived ingredient having β1,3/1,6 glucan structure, is ingested, with IL-12 inducing capability, tumor angiogenesis inhibiting capability, NK cell activating capability, and/or NKT cell activating capability used as treatment markers.

11. A commercial medium using a law of nature, on which information described in 6 to 10 is carried.

12. A commercial method utilizing the commercial medium according to 11.

### BRIEF DESCRIPTION OF THE DRAWINGS

FIG. 1 illustrates the effect on the tumor volume, with 6d indicating the results of the sixth day;

FIG. 2 illustrates the effect on the tumor volume, with 9d and 13d respectively indicating the results of the ninth and thirteenth days;

FIG. 3 illustrates the effect on the amount of IL-12 induced, with 7d indicating the results of the seventh day;

FIG. 4 illustrates the effect on the amount of IL-12 induced, with 10d and 14d respectively indicating the results of the tenth and 14^{th} days;

FIG. 5 illustrates clinical example 1;

FIG. 6 illustrates clinical example 2;

FIG. 7 illustrates clinical example 3;

FIG. 8 illustrates clinical example 4;

FIG. 9 illustrates clinical example 5;

FIG. 10 illustrates clinical example 6;

FIG. 11 is a schematic diagram showing changes in IL-12 production capability before and after IMMUTOL administration (261 examples before and 248 examples after administration);

FIG. 12 is a schematic diagram showing category-by-category changes in IL-12 production capability before and after IMMUTOL administration; “Effective”corresponds to cases in which the capability upgraded to a higher category (e.g., PR→CR, PD→NC), “No change” corresponds to cases in which the capability remained unchanged (e.g., CR→CR, NC→NC), and “Not effective” corresponds to cases in which the capability dropped to a lower category (e.g., CR→PR, NC→PD);

FIG. 13 is a schematic diagram showing changes in NK cell count (CD3−CD161+) before and after IMMUTOL administration (244 examples before and 234 examples after administration);

FIG. 14 is a schematic diagram showing changes in NKT cell count (CD3+CD161+) before and after IMMUTOL administration (261 examples before and 247 examples after administration); and

FIG. 15 is a schematic diagram showing changes in vascular endothelial growth factor (VEGF) before and after IMMUTOL administration (259 examples before and 255 examples after administration).

### DETAILED DESCRIPTION OF PREFERRED EMBODIEMENT

NBG or IMMUTOL, the main ingredient of the present invention, is a yeast having β1,3/1,6 glucan structure. The ingredient is, more precisely, trade name NBG (Norwegian Beta Glucan™) derived from saccharomyces cerevisiae or IMMUTOL (β1,3/1,6 glucan, mannose not contained, yeast cell wall protein-derived substance not contained: manufactured by ImmunoCorp). As a result of study, it was discovered that yeast having β1,3/1,6 glucan structure is a powerful IL-12 inducer and more particularly so in progressive and terminal cancer. Further, it was newly discovered that such a yeast is an anticancer composition that can hold promise of NK and NKT cell activating capabilities and tumor angiogenesis inhibiting capability by oral administration of IMMUTOL (trade name), a yeast-derived ingredient having β1,3/1.6 glucan structure.

The present inventor found that among IL-12 production inducers are not only substances such as AHCC that induce IL-12 production particularly effectively in early-stage cancer patients but also substances such as yeast-derived substances having β1,3/1,6 glucan structure according to the present invention that also deliver IL-12 production inducing effect characteristically on progressive and terminal cancer patients.

The composition or the health aid food preparations intended for oral administration of the present invention are effective for treatment against cancers such as lung cancer, lung adenoma, thymoma, thyroid cancer, bladder cancer, colon cancer, rectal cancer, caecum cancer, ureteric cancer, breast cancer, cervical cancer, brain tumor, tongue cancer, pharynx cancer, nostril cancer, larynx cancer, stomach cancer, liver cancer, bile duct cancer, testis cancer, ovary cancer, uterine body cancer, malignant melanoma and liposarcoma, but are not limited thereto. Particularly, they are preferably administered to those with low IL-12 value (e.g., 7.8 pg/ml or less) even when IL-12 production inducer such as AHCC (Aminoup) is administered.

The IL-12 production inducer, NK cell activator and NKT cell activator according to the present invention are employed in a prescription that can induce or enhance activation thereof and further maintain activation using results from immunity measurement method as indicators. That is, based on the indicators, the quantity administered and the period over which they are administered are selected for using them so as to induce or enhance activation thereof and further maintain activation. The IL-12 production inducer, NK cell activator and NKT cell activator are preferably orally ingested. Naturally, they can be parenterally ingested (including administration into vein or muscle) by reducing the quantity administered and preparing them into a quality that can endure parenteral ingestion.

The effect of IMMUTOL administration on immunity before and after the administration was measured and examined using the following markers:

(1) Angiogenesis Inhibiting Effect

Angiogenesis inhibiting effect can be judged by measuring vascular endothelial growth factor (VEGF) [-VEGF: VEGF (angiogenesis growth factor)'s opposite number (parameter value calculated by multiplying VEGF measured value by negative one)] and evaluated with negative VEGF value (-VEGF). Angiogenesis inhibiting effect can also be evaluated by using other angiogenesis growth factors such as FGF and HGF rather than VEGF value.

Evaluation is also possible with a positive value of angiogenesis inhibiting effect (e.g., endostatin) rather than VEGF.

(2) IL-12 Production Capability

Although CTL activation can be judged by CD8(+) perforin production capability, there are two CD8(+) perforin values, namely, cytotoxic T lymphocyte (CTL) and suppressor T cell (STC) The former impairs cancer cell, whereas activation of the latter results in cancer multiplication. Therefore, evaluation cannot be conducted with their absolute values. However, a cell is judged as being CTL if IFNγ is 10 IU/ml or more or if the IL-12 value is 7.8 pg/ml or more and as STC if IFNγ and IL-12 are low. For this reason, it is possible to make evaluation using IFNγ production capability (IFNγvalue) or IL-12 production capability (IL-12 value).

(3) NK and NKT Cell Activating Capabilities Both NK and NKT cells carry NKR-P1 (NK cell receptor CD161 (+)). For the former, NK cell count can be measured by the CD3 (−) CD161 (+) surface marker, whereas its activation can be judged by the CD3(−) CD161 (+) perforin production capability. For the latter, on the other hand, NKT cell count can be measured by the CD3 (+) CD161 (+) surface marker, whereas NKT cell activation can be measured by its perforin production capability.

In cancer treatment, therefore, it is possible to evaluate effector cell or angiogenesis inhibiting effect using the measurement items given below in novel immunotherapy for cancer (NITC) and common immunotherapies alike. More specifically, CTL activation can be evaluated by IFNγ or IL-12 inducing production capability. NK cell activation can be evaluated by CD3 (−) CD161 (+) or CD3 (−) CD161 (+) perforin value. NKT cell activation can be evaluated by CD3 (+) CD161 (+) or CD3 (+) CD161 (+) perforin value.

Measurement methods for cells and individual markers are illustrated below. (NKT Cell Measurement) (NK Cell Measurement) (CD8 Measurement)

Measurement of NKR-P1-bearing NKT cell can be conducted by measuring cell surface antigens (CD3 and CD161) existing specifically on the NKT cell surface. More specifically, peripheral blood lymphocyte are examined in respect of cells with positive CD3 and positive CD161 (CD3 (+) CD161 (+)). That is, CD3 and CD161, NKT cell surface antigens, are measured by the two-color test using flow cytometry with monoclonal antibody. Here, the term “NKT cell activation” refers to the fact that the CD3 (+) CD161 (+) NKT cell proportion in lymphocyte is 10% or more and preferably 16% or more. The term “NKT cell activating capability” denotes a capability of increasing the NKT cell proportion to 10% or more and preferably 16% or more, or a capability of further augmenting the NKT cell proportion more than before administration of a certain substance.

Likewise, the term “(CD3 (−) CD161 (+))” refers to examination of cell for negative CD3 and positive CD161. It has been confirmed that this method is useful for NK cell measurement in the present invention.

Further, the term “CD8 (+)” denotes examination of cell for positive CD8. It has been confirmed that this method is useful for CTL activation measurement in the present invention.

In examples, using cancer patients' blood, blood cell were discriminated as positive or negative for cell surface antigens such as CD3, CD161 and CD8, with each cell proportion measured, as normally done, by the two-color test using flow cytometry. At this time, each of monoclonal antibodies for CD3, CD161 and CD8 was manufactured by Coulter or Becton Dickinson.

(Perforin Production Cell Measurement)

Two among CD3, CD8 and CD161, cell surface antigens, and perforin are measured in respect of peripheral blood lymphocyte as normally done by the two-color test using flow cytometry. More specifically, sampled blood is added with fixative, thus fixing cells. After addition of membrane permeating solution, anti-perforin antibody (manufactured by Pharmingen) is added for reaction. Further, PRE-Cy5 labeled second antibody (manufactured by DAKO) is added for reaction, followed by addition of anti-CD3-PE (Coulter 6604627) antibody and anti-CD161-FITC (B-D) antibody for reaction, after which measurement is made by flow cytometry. Abbreviations in the figures are indicated as PERF. (Sample Preparation for Cytokine Measurement)

First, mononuclear cell fraction is isolated from blood for preparation. Heparin-added peripheral blood is diluted twofold with phosphate buffered saline (PBS) and mixed, then the mixture is layered over Ficoll-Conray solution (specific gravity: 1.077), centrifuging at 400G for 20 minutes, after which mononuclear cell fraction is collected. After washing, 10% fetal bovine serum (FBS)-added RPMI-1640 culture medium is added for preparation to provide a cell count of 1×10⁶. Phytohemagglutinin (manufactured by DIFCO) is added to 200 μl of the cell suspension thus obtained to provide a concentration of 20 μg/ml, and then the mixture is cultured using a 96 well micro plate under 5% Co₂ at 37° C. for 24 hours. The cell solution thus cultured is used as the Cytokine measurement sample. (IL-12 Amount Measurement)

The amount of IL-12 induced can be measured using a measurement kit based on the enzyme-linked immuno sorbent assay (ELISA) without making indirect measurement as is done with human since a sufficient amount of IL-12 is induced in serum in the experimental examples using mice described below. In this experimental system using mice, it is possible to examine for IL-12 production inducing capability by having them orally ingest IL-12 production inducing substance continuously and finding increase in amount of blood IL-12 thereafter.

It is to be noted that the amount of blood IL-12 is not directly measurable in humans due to existence of inhibitor in blood. For instance, measurement of the amount of IL-12 induced in a cancer patient is conducted using a cultured solution made available by first feeding a stimulant to peripheral blood mononuclear cell isolated and prepared from the cancer patient's blood, culturing the mixture and centrifuging it for cell removal. The number of cells subjected to culture is 0.5×10⁶ cell/ml to 1×10⁷ cell/ml and preferably 1×10⁶ cell/ml. For cell-stimulating substance, meanwhile, phytohemagglutinin (PHA) —a conventionally used mitogen —is added to provide a final concentration of 0.1 to 100 μg/ml and preferably 1 to 20 μg/ml for culture. Cell-stimulating substance is not limited to PHA, and any substance may be used as long as the substance is capable of stimulating cells and thus causing them to produce immunobiological active substance in order to achieve the objects of the present invention. PMA (Phorbol 12-Myristate-13-Acetate), PMA+Ionomycin, LPS (Lipopolysaccharide) and PWM (Poke Weed Mitogen) are included in such substances. While IL-12 amount measurement itself may be performed using publicly known clinical or biochemical tests, a measurement kit available from R&D SYSTEMS or MBL is used that is based on the enzyme-linked immuno sorbent assay (ELISA). Here, the term “IL-12 production inducing capability” refers to a capability of augmenting the amount of IL-12, which is produced by peripheral blood mononuclear cell as a result of stimulation, to 7.8 pg/ml or more, or a capability of augmenting the amount of IL-12 produced more than before administration of a certain substance.

(Angiogenesis Inhibiting Capability Measurement)

(Measurement of Vascular Endothelial Growth Factor, VEGF and Basic Fibrolast Growth Factor, bFGF and Angiogenesis Inhibiting Factor Endostatin, endostatin)

In-serum concentrations were measured using individual enzyme immunoassay solid-phase techniques in commercial kits (ELISA: enzyme linked immuno sorbent assay) (ACCUCYTE Human VEGF, ACCUCYTE Human bFGF, ACCUCYTE Human Endostatin: CYTIMMUNE Sciences Inc.).

The composition intended for oral ingestion according to the present invention comprises, as an active ingredient having IL-12 production inducing capability, NBG or IMMUTOL each of which is a yeast-derived ingredient having β1,3/1,6 glucan structure.

The composition intended for oral ingestion according to the present invention comprising NBG or IMMUTOL as an active ingredient for inducing IL-12 production, each of which is a yeast-derived ingredient having β1,3/1,6 glucan structure, differs considerably from AHCC that is publicly known for its IL-12 production inducing capability in individual stages of cancer progression. The composition according to the present invention whose effective ingredient is a yeast-derived ingredient having β1,3/1,6 glucan structure exhibits a sufficient IL-12 production inducing capability in the initial stage of cancer and characteristically delivers an equivalent or more powerful IL-12 production inducing capability in progressed terminal cancer. On the other hand, while AHCC delivers a characteristic IL-12 production inducing capability in the initial stage of cancer, its inducing capability falls off as the cancer progresses.

The amount of administration of the composition intended for oral ingestion according to the present invention is 1 to 2000 mg/kg of body weight per day and preferably about 10 to 2000 mg/kg of body weight, with the composition preferably orally ingested one to several times per day over the time period of 10 days to one year. Naturally, the composition can be parenterally ingested by reducing the amount administered and preparing it into the quality such as that administration via parenteral ingestion can be endured.

NBG or IMMUTOL, the main ingredient of the present invention and a yeast-derived ingredient having β1,3/1,6 glucan structure each, is publicly known as food material. For instance, NBG or IMMUTOL (ImunoCorp) and the like are illustrated. It is to be noted that commercial products were used as samples in the present invention.

Oral preparations are prepared into tablets, powders, capsules, syrups, etc. Preparations can naturally be produced as such by mixing them with a necessary additive such as known filler, disintegrator, binder or lubricant through stereotyped means. Further, flavoring agent, colorant, perfume, stabilizer, disinfectant, antiseptic and the like may be added as necessary.

As described above, the present invention clarifies the relationship between the composition intended for oral ingestion comprising NBG or IMMUTOL, a yeast-derived ingredient having β1,3/1,6 glucan structure each, as effective ingredient, and the IL-12 production inducing capability during progression stages of cancer and also clarifies the relationship between administration of IMMUTOL (trade name), a yeast-derived ingredient having β1,3/1,6 glucan structure, and IL-12 production capability augmentation, angiogenesis inhibiting effect, NK cell activation and/or NKT cell activation. A commercial medium carrying these pieces of information serves asmeans for differentiating values of the product. Therefore, the commercial medium carrying these pieces of information is extremely high in usefulness. Additionally, since these pieces of information, if used commercially, serve as means for differentiating the product values, a commercial method using these pieces of information is extremely high in usefulness.

Information such as that described above, if carried on a medium using a law of nature, serves as a useful commercial medium, and the commercial medium provides a useful commercial method.

### Example

While a detailed description will be given below of the present invention with reference to examples, the present invention is not limited thereto.

### Example 1

Immunological antitumor effect and IL-12 production capability were examined using Saccharomyces cerevisiae having β1,3/1,6 glucan (Alpenrose Saccharomyces cerevisiae (300 mg/kg) and Norwegian-produced Saccharomyces cerevisiae extract (NBG 10 mg/kg)). It is to be noted that each was administered in adjusted amounts such that the β1,3/1,6 glucan content in both groups became identical to each other.

In the experiment, 2×10⁶ cells/mouse of 3LL tumor were transplanted to B10 mice (C57BL/10), with a comparison made in terms of tumor volume and IL-12 production on the 13^{th} day.

After the tumor transplantation, the tumor volume in control (A) for forced oral administration of water was 239.41±150 mm³, whereas an increasing tendency was observed in the tumor volume for normal Saccharomyces cerevisiae/dry Saccharomyces cerevisiae (B) (300 mg/kg) as compared with the control.

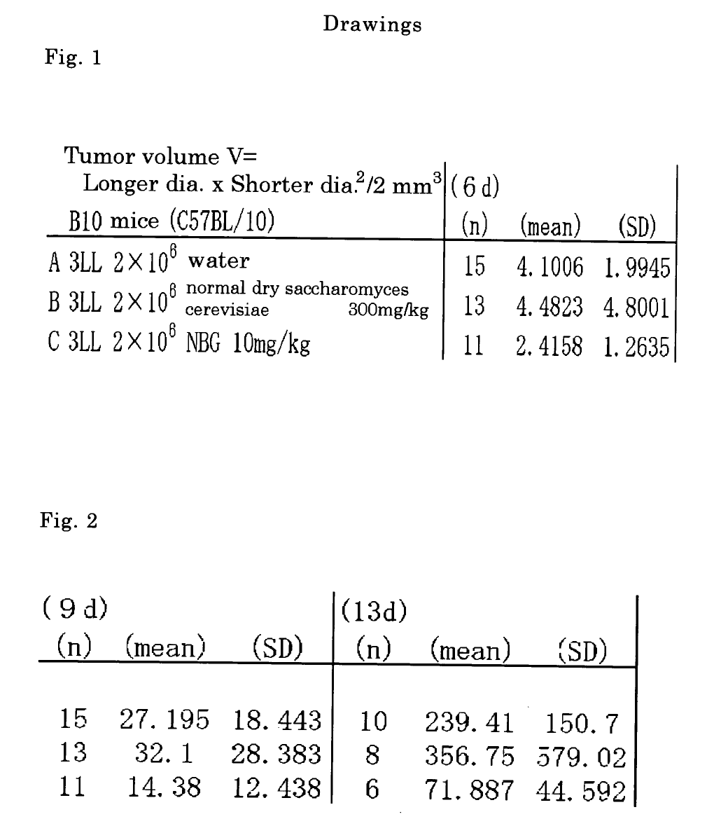

In an case in which NBG (10 mg/kg) was administered, the tumor volume was found to have significantly shrunk (71.887±44.592 mm³) (P<0.0393). FIGS. 1 and 2.

As for IL-12 concentration in blood, NBG (10 mg/kg) showed a significant increase in IL-12 concentration relative to control (A). IL-12 concentration was measured with Biotrak RPN2702 Interleukin-12total{(m) IL-12}, (p40andp70), mouse ELISA system kit from Amersham Pharmacia.

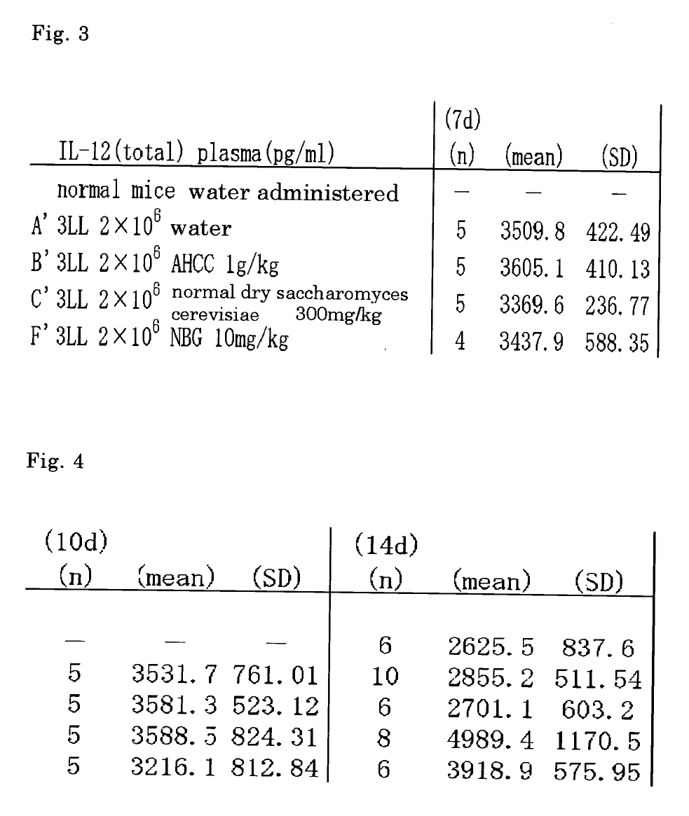

That is, as compared with control (A′) for forced water oral administration, IL-12 showed an elevated value in all examination groups, with a significant increase in IL-12 concentration for NBG (FIGS. 3 and 4).

### Clinical Examples

While a specific description will be given below of the present invention with reference to clinical examples, the present invention is not limited thereto. It is to be noted that the efficacy of therapies used was rated as Complete Remission (CR), Partial Remission (PR), No Change (NC) or Progressive Disease (PD) in accordance with the Standard for judgment of the efficacy of anticancer agent under GCP of the Japan Ministry of Health and Welfare.

### Clinical Example 1

I.Y. 60 y.o. Rectal Cancer Metastasized to Liver and Lung

The patient has rectal cancer metastasized to the liver and the lung. Administration of β1,3 glucan from mushroom mycelium began on Sep. 12, 200X, with 6.0 g/day of Better Shark MC and ILX (registered trademarks), 3.0 g/day of ILY (registered trademark) and 3.0 g/every other day of krestin. This treatment method is named “NITC” by Yagita. For the five-month period from the start of NITC, the patient remained in NC, but into the sixth month of the administration nearly all tumor markers increased.

For this reason, administration of 6 T/day of IMMUTOL, a yeast-derived β1,3 glucan, began. In two months after IMMUTOL administration started, IFNγ and IL-12 showed a noticeable increase, with tumor markers initiating a decline. In the third month, tumor markers improved further, maintaining the patient in PR.

The present case also indicates that additional administration of yeast β1,3 glucan is able to lead to tumor shrinkage in patients rated as NC or PD by mushroom mycelium.

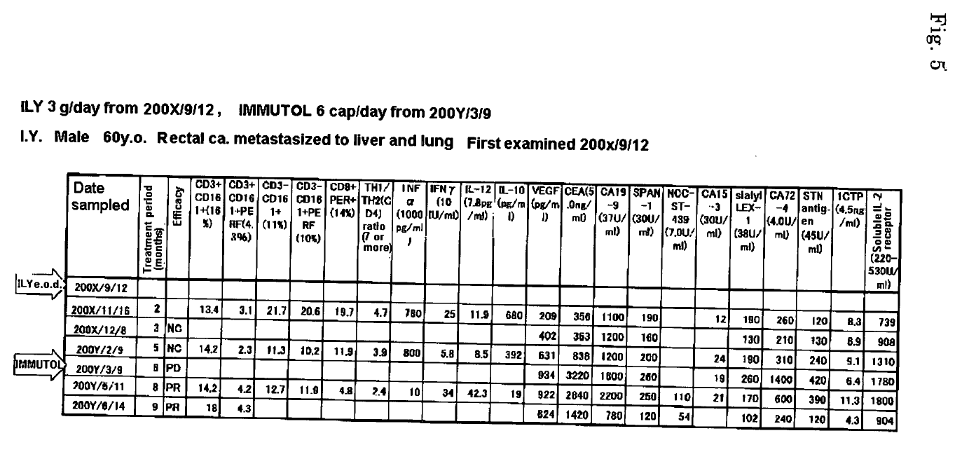

Detailed data is shown in FIG. 5.

### Clinical Example 2

N.H. 56 y.o. Breast Cancer Metastasized to Axillary Lymph Node

The patient, a 50-year-old woman, had her right breast and the metastasized lesion of the axillary lymph node removed due to breast cancer on Jul. 21, 200& (at Cancer Institute). Liver metastasis emerged on Nov. 200&, and she had the liver portion removed on Mar. 26, 200X. NITC began on Apr. 20, 200X. A number of metastases to the removed end of the liver and to the lungs were observed on Jun. 12, 200X. For this reason, administration of 3.0 g/every other day of ILY began. since Jul. 13, the administration was changed to 3.0 g/day of ILY. On Aug. 4, one month later, some tumor markers showed a decline, namely, 773 ng/ml for CEA, 13 U/ml for NCC and 7.7 ng/ml for ICTP. Because HER-2 was found to be (+++), weekly administration of (paclitaxel+herceptin) started on Aug. 7 at Cancer Institute. From then, CEA successfully slipped, reaching 4.3 ng/ml on Nov. 200X, whereas SLX-1 also dropped to 30 U/ml, both of which are normal levels, as a result of which the patient was rated as CR. CEA, however, increased gradually from then, and therefore taxol was stopped, thus resulting in administration of herceptin alone.

To reinforce immunity activating capability, 4 T/day of IMMUTOL began on Mar. 23, 200Y.

Then, while the CEA value shot up to 81.8 ng/ml one month later, it dropped to 55.7 ng/ml two month later and further declined to 27.4 ng/ml in three months, as a result of which the patient was rated as PR.

ILY was administered in addition to 6.0 g/day of ILX and 3.0 g/every other day of krestin, both mushroom myceliums, and further taxol and herceptin were administered additionally, considerably reducing the tumor markers and thereby resulting in the patient being temporarily rated as CR. However, the markers shot up again since Jan. 200Y, resulting in the patient being rated as PD.

For this reason, 4 T/day of IMMUTOL, a yeast-derived β1,3 glucan, was additionally administered, leading to a noticeable decline in tumor markers and resulting in the patient being rated as PR.

This fact was presumably thanks to immunity activating capability of yeast β1,3 glucan.

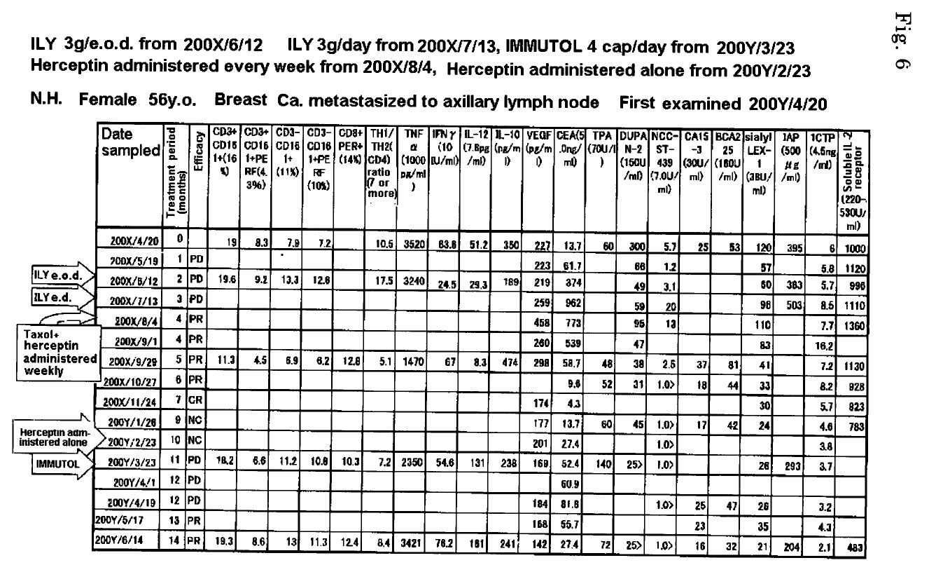

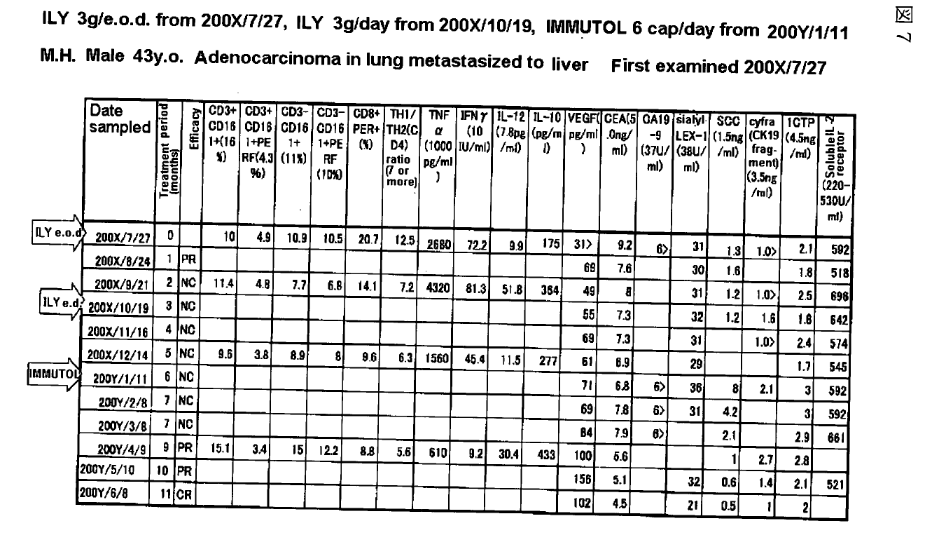

Detailed data is shown in FIG. 6.

### Clinical Example 3

M.H. 43 y.o. Adenocarcinoma in Lung Metastasized to Liver

The patient, suffering adenocarcinoma in the right lung with three metastases to the liver, visited the hospital, hoping for immunotherapy. The patient began taking Better Shark MC, an angiogenesis inhibitor, and ILX (6.0 g/day), ILY (3.0 g/day) and krestin (3.0 g/every other day), mushroom mycelium β1,3 glucans, on Jul. 27, 200X. CEA, one of the tumor markers, was found to be high or 9.2 ng/ml, with three metastases to the liver and a 3 cm-large adenocarcinoma in the right lung demonstrated by echo test and breast CT. While the IL-12 value increased with the CEA value showing a declining tendency until Sep. 21, SCC showed an abnormal value or 8.0 on Jan. 11, 200Y, as a result of which administration of yeast IMMUTOL (6 T/day) began. Then, the CEA and SCC values continued their decline. On Jun. 8, 200Y, all tumor markers showed a normal value, and metastases to the liver and the adenocarcinoma in the lung vanished, resulting in the patient being rated as CR. This fact demonstrates that while ILX, ILY and krestin, mushroom myceliums, stopped the progress of the tumors but did not provide a sufficient immunity activation, administration of a yeast-derived substance (β1,3 glucan) is able to guide the patient from NC to CR.

Detailed data is shown in FIG. 7.

### Clinical Example 4

T.K. 66 y.o. Male, Adenocarcinoma in Lung

The patient, a 66-year-old male and diagnosed with adenocarcinoma in the lung, began NITC (6.0 g/day of ILX, 3.0 g/day of ILY, 20 g/day of Better Shark LO) on Oct. 3, 200X.

One month later, CEA (normal not more than 5.0 ng/ml) shot up to 13.2 ng/ml but remained constant thereafter until the sixth month.

However, the CEA value began rising from the ninth month over a period of ten months, during which the patient was rated as PD.

For this reason, oral administration of six capsules of IMMUTOL (1.2 g)/day divided by three, a yeast preparation, began.

Thereafter, the CEA value continued its gradual decline, maintaining high IL-12 production capability, increasing the NK cell (CD3-CD161+) and NKT cell (CD3+CD161+) counts and augmenting its activity.

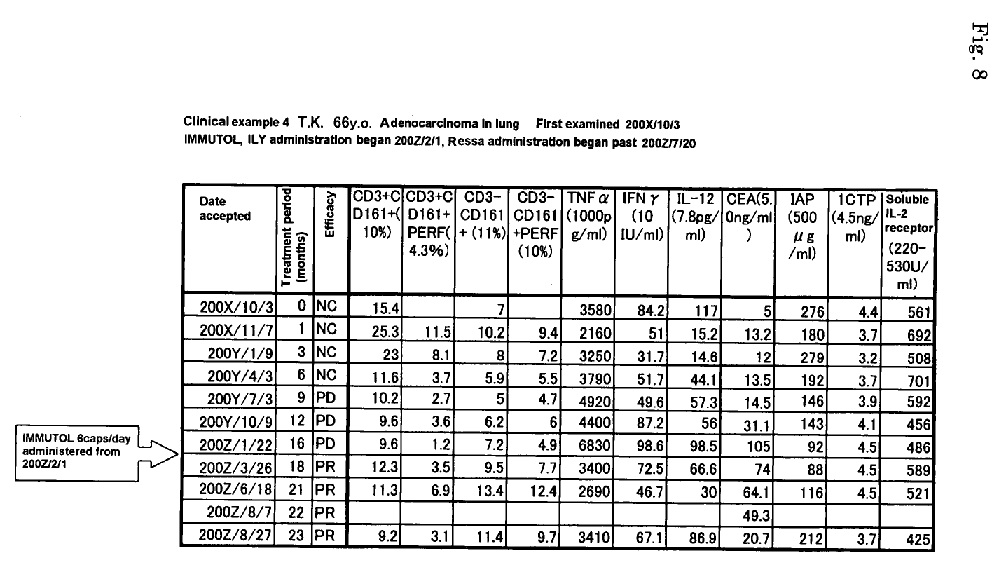

Detailed data is shown in FIG. 8.

### Clinical Example 5

I.Y. 72 y.o. Male, Adenocarcinoma in Lung

This is a case of a 72-year-old male with adenocarcinoma in the lung.

NITC (6.0 g/day of ILX, 3.0 g/day of ILY, 20 g/day of Better Shark LO) started on Jun. 3, 200X.

For the following five months, all tumor markers including CEA gradually increased, leading to the patient being rated as PD.

Until the ninth month thereafter, the markers remained unchanged, causing the patient to be rated as NC. For the following four months, the patient was rated as PR and then rated as PD from the 14^{th} to 20^{th} months.

Since the tumor markers showed an increase despite additional administration of three capsules of IMMUTOL (0.6 g)/day divided by three from the 20^{th} month, IMMUTOL was increased to six capsules (1.2 g)/day divided by three.

Thereafter, the tumor markers are continuing their decline.

The optimal amount of IMMUTOL is presumably six capsules rather than three capsules.

As for immunity, augmentation of IL-12 production capability was observed by increasing amount of IMMUTOL.

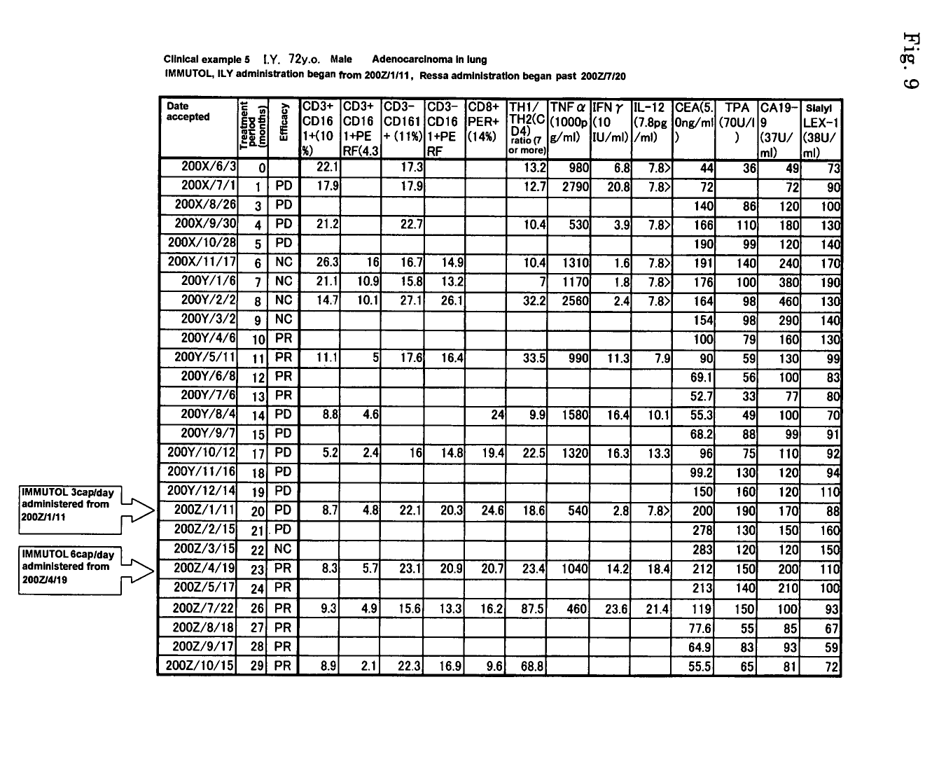

Detailed data is shown in FIG. 9.

### Clinical Example 6

T.F. 52 y.o. Female, Ascending Colon Cancer

The patient is a 52-year-old woman with ascending colon cancer metastasized to the liver and the lung, and NITC (6.0 g/day of ILX, 3.0 g/day of ILY, 20 g/day of Better Shark LO) began on May. 21, 200Y.

While the patient remained in NC for six months, the tumor markers rose during the eighth and ninth months, causing her to be rated as PD.

For this reason, administration of six capsules of IMMUTOL (1.2 g)/day divided by three started.

Into the second month after administration of six capsules of IMMUTOL/day, NK cell count and its activity were maintained at the same time that IL-12 production capability rose. CEA, Cal9-9 and Span-1 all fell within normal range, resulting in the patient being rated as CR.

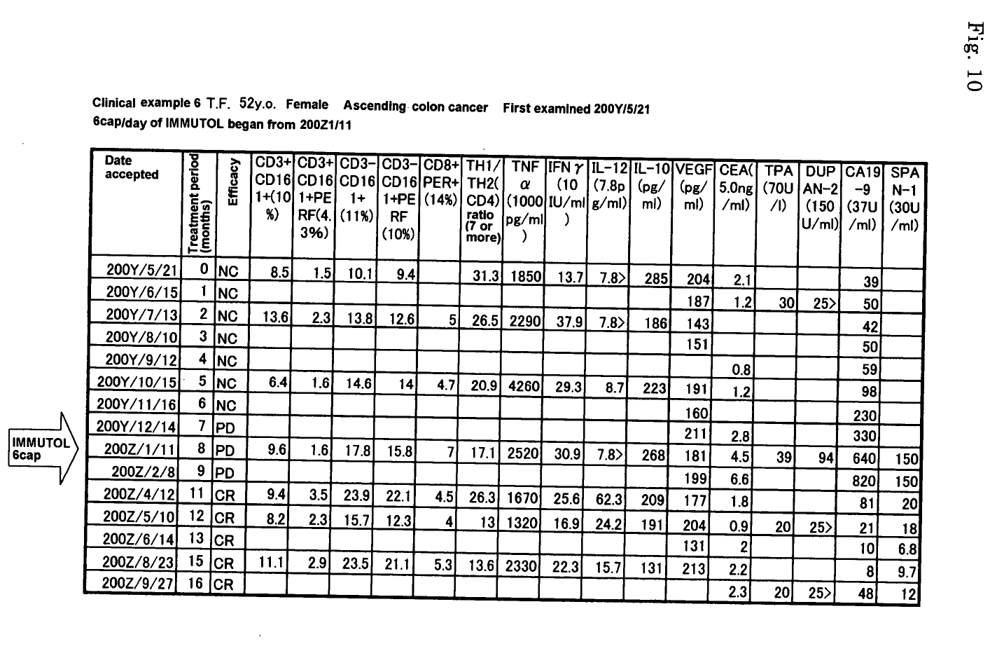

Detailed data is shown in FIG. 10.

### Experimental Example

Effect of IMMUTOL Administration on Immunity

Changes in marker values were measured and examined before and after IMMUTOL administration in the same manner as with the clinical examples. IL-12 production capability was evaluated on a category-by-category basis (effect-by-effect by IMMUTOL administration).

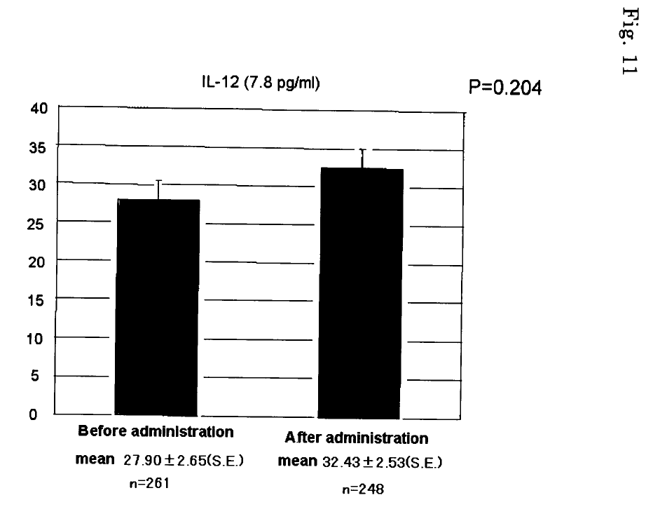

FIG. 11 shows changes in IL-12 production capability before and after IMMUTOL administration (261 examples before and 248 examples after administration). While IL-12 production capability exhibited, as a whole, an upward tendency (p=0.204), with the post-treatment value of 32.43±2.53 pg/ml compared to the pre-treatment value of 27.90±2.65 pg/ml, the difference was not recognized as significant one.

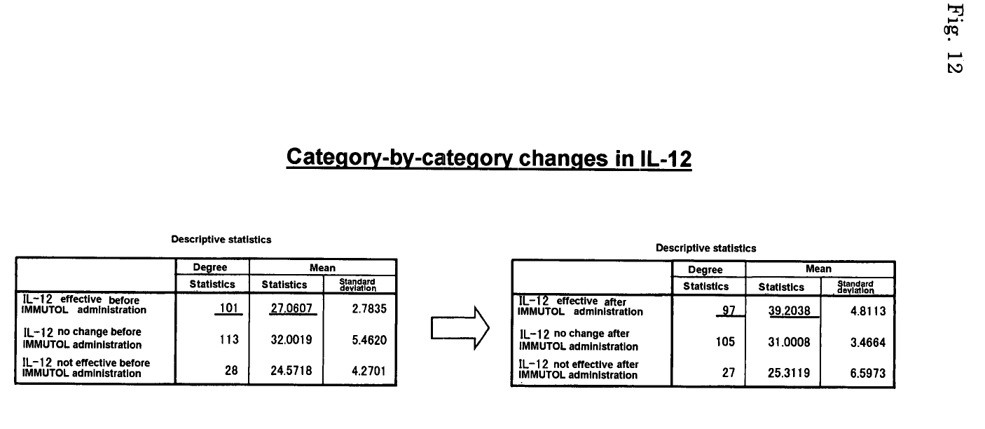

FIG. 12 illustrates category-by-category changes in IL-12 production capability before and after IMMUTOL administration. In the effective group (101 examples; improved by IMMUTOL administration), the post-treatment value was increased significantly to 39.2038±4.8113 pg/ml as compared with the pre-treatment value of 27.0607±2.7835 pg/ml (P<0.01).

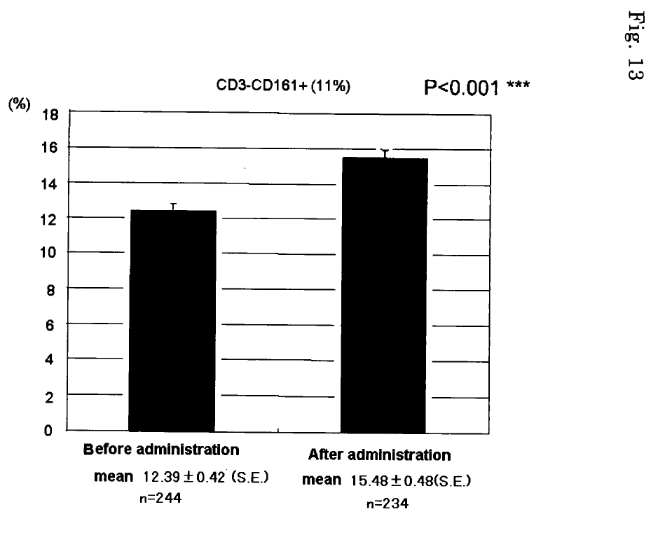

FIG. 13 shows changes in NK cell count before and after IMMUTOL administration (244 examples before and 234 examples after administration). A significant improvement was observed with the post-treatment value of 15.48±0.48% as compared with the pre-treatment value of 12.39±0.42% (p<0.001).

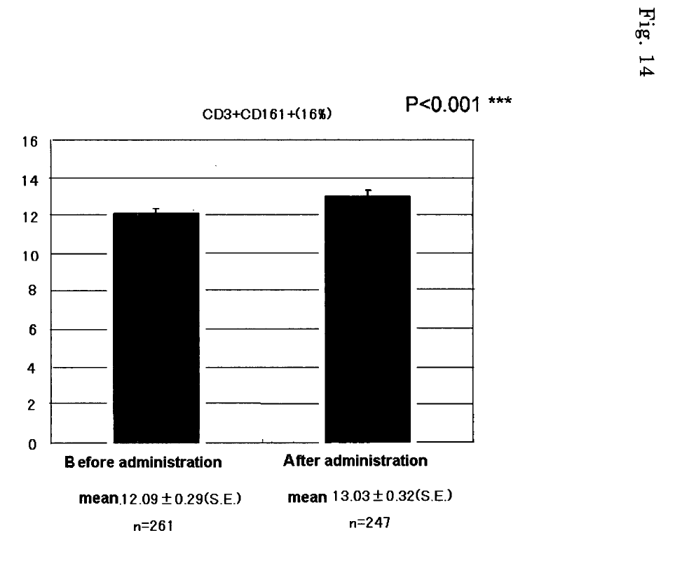

FIG. 14 illustrates changes in NKT cell count before and after IMMUTOL administration (261 examples before and 247 examples after administration). A significant improvement was observed with the post-treatment value of 13.03±0.32% as compared with the pre-treatment value of 12.09±0.29% (p<0.001).

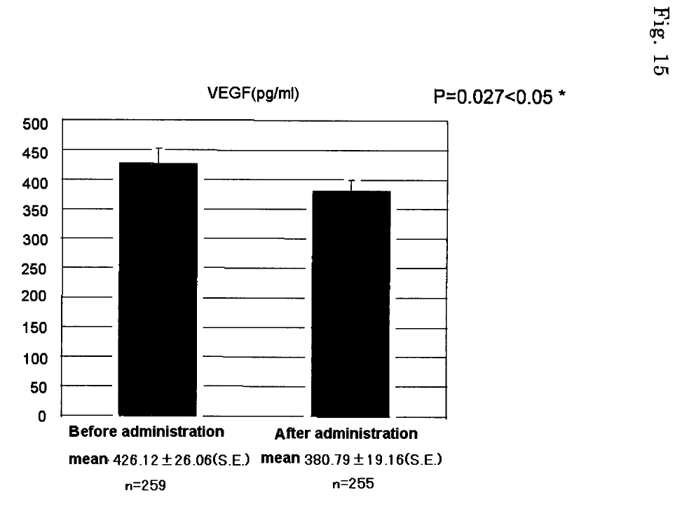

FIG. 15 shows changes in vascular endothelial growth factor (VEGF) before and after IMMUTOL administration (259 examples before and 255 examples after administration). VEGF declined significantly from 426.12±26.06 pg/ml before administration to 380.79±19.16 pg/ml after administration (p<0.05).

From the above findings, it was verified that administration of IMMUTOL (trade name), a yeast-derived ingredient having β1,3/1,6 glucan structure, is clinically effective. Additionally, correlation was observed between administration of IMMUTOL (trade name), a yeast-derived ingredient having β1,3/1,6 glucan structure, and augmentation of IL-12 production, angiogenesis inhibiting capability, NK cell activating capability and/or NKT cell activating capability. As a result, it was confirmed that ingestion of IMMUTOL (trade name), a yeast-derived ingredient having β1,3/1,6 glucan structure, is effective for cancer treatment using IL-12 inducing capability, angiogenesis inhibiting capability, NK cell activating capability and/or NKT cell activating capability as treatment markers.

### Industrial Applicability

The anticancer composition according to the present invention was successfully provided by newly finding the fact that a composition comprising NBG or IMMUTOL, a yeast-derived ingredient having β1,3/1,6 glucan structure each, is an unprecedentedly effective IL-12 inducer and further discovering that the composition can hold promise of NK and NKT cell activating capabilities and tumor angiogenesis inhibiting capability as a result of oral administration of IMMUTOL (trade name), a yeast-derived ingredient having β1,3/1,6 glucan structure.

1. An IL-12 inducer comprising NBG or IMMUTOL (trade name).

2. An NK cell and/or NKT cell activator comprising NBG or IMMUTOL (trade name).

3. (canceled)

4. (canceled)

5. (canceled)

6. A treatment drug for progressive or terminal cancer comprising NBG or IMMUTOL (trade name), wherein the form of administration is oral.

7. A treatment drug for progressive or terminal cancer, wherein NBG or IMMUTOL (trade name) is ingested, with IL-12 inducing capability as treatment marker.

8. A treatment drug for progressive or terminal cancer, wherein NBG or IMMUTOL (trade name) is ingested, with NK cell activating capability and/or NKT cell activating capability as treatment marker.

9. A treatment drug for progressive or terminal cancer, wherein NBG or IMMUTOL (trade name) is ingested, with angiogenesis inhibiting capability as treatment marker.

10. A treatment drug for progressive or terminal cancer, wherein NBG or IMMUTOL (trade name) is ingested, with IL-12 inducing capability, tumor angiogenesis inhibiting capability, NK cell activating capability and/or NKT cell activating capability as treatment markers.

11. A commercial medium using a law of nature, on which information described in claim 6 is carried.

12. A commercial method utilizing the commercial medium according to claim 11.

13. An IL-12 inducer comprising NBG or IMMUTOL (trade name), wherein the inducer is administered to progressive or terminal cancer patients.

14. NK cell and/or NKT cell activator comprising NBG or IMMUTOL (trade name), wherein the activator is administered to progressive or terminal cancer patients.

15. The inducer according to claim 13, wherein the progressive or terminal cancer:
1) rectal cancer,
2) hepatoma,
3) lung cancer,
4) breast cancer,
5) adenocarcinoma in lung, or,
6) liver cancer.

16. The activator according to claim 14, wherein progressive or terminal cancer:
1) rectal cancers,
2) hepatoma,
3) lung cancers,
4) breast cancers,
5) adenocarcinoma in lung or,
6) liver cancer.

17. The inducer according to claim 13, wherein NBG or IMMUTOL is ingested orally in vivo from 10 to 2000 mg/kg of body weight/day in amounts.

18. The activator according to claim 14, wherein NBG or IMMUTOL is ingested orally in vivo from 10 to 2000 mg/kg of body weight/day in amounts.

19. A cancer treatment method, comprising:
measuring IL-12 inducing capability first,
administrating NBG or IMMUTOL (trade name) to a patient, and
measuring again IL-12 inducing capability.

20. The inducer according to claim 15, wherein NBG or IMMUTOL is ingested orally in vivo from 10 to 2000 mg/kg of body weight/day in amounts.

21. The activator according to claim 16, wherein NBG or IMMUTOL is ingested orally in vivo from 10 to 2000 mg/kg of body weight/day in amounts.
